(19) United States
(12) Patent Application Publication
(10) Pub. No.: US 2008/0044830 A1
(43) Pub. Date: Feb. 21, 2008

(54) Three-Dimensional Nanostructured and Microstructured Supports

(75) Inventors: Gunter Tovar, Stuttgart (DE); Herwig Brunner, Stuttgart (DE); Achim Weber, Altbach (DE); Kirsten Borchers, Stuttgart (DE)

Correspondence Address: DICKSTEIN SHAPIRO LLP, 1177 AVENUE OF THE AMERICAS (6TH AVENUE), NEW YORK, NY, 10036-2714, US

(73) Assignees: Fraunhofer-Gesellschaft zur Forderung der Angewandten Forschung E.V., Munchen (DE); Universitat Stuttgart, Stuttgart (DE)

(21) Appl. No.: 11/722,492

(22) PCT Filed: Dec. 2, 2005
(86) PCT No.: PCT/EP05/12972

§ 371 Date: Jun. 21, 2007

(30) Foreign Application Priority Data
Dec. 24, 2004 (DE) 10 2004 062 573.5

(51) Int. Cl. G01N 33/543; C12N 1/00; C12Q 1/00; C12Q 1/04; H05H 1/24; G01N 21/00; G01N 33/53

(52) U.S. Cl. 435/6; 427/195; 427/569; 435/307.1; 435/34; 435/375; 435/4; 436/164; 436/501

(57) ABSTRACT

The invention relates to functional elements that comprise, disposed on a support, microstructures containing biofunctionalized nanoparticles, methods for producing these functional elements and use of the same.

The present invention relates to functional elements, comprising microstructures made of nanoparticles that can be biofunctionalized or are biofunctionalized and disposed on a carrier, and to methods for producing these functional elements as well as to the use thereof.

The role played by analysis of biological molecules such as DNA or proteins is becoming increasingly important in a wide variety of fields, for example in environmental analysis for the detection of microorganisms, in clinical diagnostics for the identification of pathogens or for the determination of drugs resistance and the like. Regardless of the specific application, these analytical methods must always meet the same requirements. In particular, they should be quick and cost-efficient to implement, and at the same time they must provide high sensitivity and reliably reproducible results.

WO 03/056336 A2, for example, describes the production and use of microstructures made of biofunctionalized nanoparticles, which can be used in a wide variety of detection and analytical methods.

The microstructures described, however, leave room for improvement in terms of sensitivity, particularly in cases where a high level of detection accuracy is required.

The present invention is therefore directed towards the technical problem of developing means and methods for producing miniaturized carrier systems with biological molecules immobilized thereon, for example gene chips and protein chips, wherein the disadvantages known from the state of the art have been eliminated; more particularly, even greater detection sensitivity is provided and the biomolecules are, or can be, immobilized at high packing densities on a carrier, particularly while preserving and protecting biological activities thereof, these means and methods being suitable for applications in a wide variety of screening and analytical systems, such as in medical measurement and monitoring technologies, and in biocomputers.

The present invention solves the underlying technical problem by providing a functional element comprising a carrier having a surface and at least one microstructure on the carrier surface, wherein the microstructure is formed by a plurality of layers of nanoparticles disposed three-dimensionally on top of each other and wherein the nanoparticles have molecule-specific recognition sites, which allow addressability within the microstructure.

The invention thus provides multidimensionally configured microstructures comprising a plurality of nanoparticle layers, which in a preferred embodiment are formed by the inclusion of at least one biomolecule-stabilizing agent. These multidimensionally configured layers of nanoparticles markedly expand the reaction surfaces of the functional elements, these surfaces being provided for the desired detection reaction; and in a preferred embodiment, by further including the protein-stabilizing agent, it is possible to preserve the natural structure and function of the element when using proteins or peptides as biologically active molecules.

Surprisingly, it was demonstrated that the plurality of three-dimensionally configured layers of nanoparticles, particularly at a thickness measuring 10 nm to 10 μm, preferably 50 nm to 2.5 μm, and particularly 100 nm to 1.5 μm, remain disposed on the surface of the carrier in a stable fashion, even if they are exposed to extended and vigorous rinsing and/or washing steps. The three-dimensionally configured plurality of layers of nanoparticles provided according to the invention remains surprisingly stable on the carrier surface and thus, contrary to expectations, enables a high level of detection accuracy, even at micro-quantities of analytes to be detected. The stability of the three-dimensional microstructures made of nanoparticles provided according to the invention enables the production of functional layers, which can bind specific analytes in a localized manner. As a result, the three-dimensional microstructured nanoparticle layers meet all requirements associated with their application in the production of biological sensor surfaces.

The present invention thus provides a functional element, the surface of which has one or more microstructures, each microstructure comprising a plurality of nanoparticles in a plurality of layers with identical or non-identical molecule-specific recognition sites. The microstructures of the functional elements may have, or may be provided with, biofunctions. This means that the molecule-specific recognition sites of the nanoparticles forming the microstructure are able to detect and bind appropriate molecules, particularly organic molecules having a biological function or activity. These molecules can be nucleic acids or proteins, for example. Further molecules, for example molecules to be analyzed in a sample, can then be bound to other molecules that are bound to the molecule-specific recognition sites of the nanoparticles. Unlike systems known from the state of the art, for example conventional gene or protein arrays, the present invention also provides for the binding of biological molecules not directly to a planar surface, but rather so as to be immobilized on a plurality of three-dimensional nanoparticle surfaces, which are used prior to, or following, immobilization to form a microstructure.

Compared to conventional systems, for example such as those which immobilize the biological molecules directly on the carrier, the functional elements according to the invention, wherein the elements comprise nanoparticulate systems with molecule-specific recognition sequences for the binding of biological molecules, offer several crucial advantages.

The nanoparticles used according to the invention are extremely flexible, inert systems. They may have, for example, a wide variety of cores, such as organic polymers or inorganic materials. Inorganic nanoparticles such as silica particles have the advantage that they are notedly chemically inert and mechanically stable. While surfmers and polymers subjected to molecular imprinting have soft cores, nanoparticles with silica or iron cores undergo no swelling in solvents. Non-swellable particles do not alter their morphology, even if they are suspended in solvents repeatedly over extended periods. Functional elements according to the invention that comprise non-swellable particles can therefore be used without difficulty in analytical or microstructuring methods that require the use of solvents, without adversely affecting the state of the nanoparticles or the immobilized biological molecules. Functional elements having such nanoparticles can thus also be used for the purification of the biological molecules to be immobilized from complex substance mixtures that comprise undesirable substances, such as detergents or salts, as the molecules to be immobilized can be optimally separated from such substance mixtures through washing processes of any desired duration. On the other hand, super-paramagnetic or ferromagnetic nanoparticles having an iron oxide core can align in a magnetic field along the field lines. This characteristic of iron oxide nanoparticles can be exploited to directly create microstructures, particularly nanoscopic printed circuit board tracks.

The functional elements according to the invention may be used for immobilizing a wide variety of biological molecules, while preserving the biological activity of the molecules. The nanoparticles used for forming the microstructures have molecule-specific recognition sites, particularly functional chemical groups, which can bind the molecule to be immobilized so that the molecule regions required for the biological activity are present in a state that corresponds to the native state of the molecule. As a function of the functional groups present on the nanoparticle surface, the biomolecules may be covalently or non-covalently bound to the nanoparticles, as needed. The nanoparticles may comprise different functional groups, so that either different biomolecules or biomolecules with different functional groups can be immobilized in a preferred orientation. The biomolecules can be immobilized on the nanoparticles either oriented or unoriented, with nearly any desired orientation of the biomolecules being possible. As a result of the immobilization of the biomolecules on the nanoparticles, stabilization of the biomolecules is also achieved.

According to the invention, it is preferable that at least one biomolecule-stabilizing agent, and particularly at least one protein-stabilizing agent, be enclosed in the microstructure. Such agents further enhance the stabilization of the biomolecules. The addition of at least one biomolecule-stabilizing additive, and particularly at least one protein-stabilizing additive, preserves the functionality of biological molecules that are bound to the nanoparticles, and particularly the functionality of peptides or proteins, within the particle layers when these are dried on a substrate, and thus ensures that the nanoparticulate functional layers have a good shelf life. The shelf life can be up to one year, preferably up to 8 months, and particularly 3 months. The inclusion according to the invention of at least one biomolecule-stabilizing agent, and particularly at least one protein-stabilizing agent, in the microstructure thus protects the function, particularly the biological function, and effectiveness of the functional elements according to the invention.

The nanoparticles used to form the microstructures have a comparatively large surface area-to-volume ratio and are accordingly able to bind a large amount of a biological molecule per mass. In comparison with systems where biological molecules are bound directly to a planar carrier, a functional element can thus bind a considerably larger amount of biological molecules per unit of area. The great quantity of molecules bound per unit area, which is to say, the packing density, according to the invention, is because a plurality of particulate layers are disposed on top of each other in order to create the microstructure on the carrier surface. The quantity of biological molecules bound per unit area can be further increased by coating the nanoparticles first with hydrogels and then with biological molecules.

The production and use of a plurality of three-dimensionally disposed layers of nanoparticles according to the invention expands the reactive surface, as compared to previously used planar affinity surfaces. As a result, the microstructures according to the invention are able to bind more analyte. The more nanoparticles are immobilized per area, the more analyte the nanoparticle multilayers bind. The analyte concentrations and the signal intensity that are achieved after the analyte binds to the nanoparticulate surfaces are linearly correlated.

The nanoparticles used according to the invention have a diameter ranging from 5 nm to 500 nm. Therefore, when using such nanoparticles, functional elements can be produced, which have very small microstructures of arbitrary shape in the nanometer to micrometer range. The use of the nanoparticles for producing the microstructures thus allows an unprecedented miniaturization of the functional elements, which is associated with considerable improvements in crucial parameters of the functional elements.

The nanoparticles used according to the invention exhibit excellent adhesion properties on the materials that are used for producing the carriers and/or carrier surfaces. As a result, the particles can be used without difficulty for a variety of carrier systems and hence for a variety of different functional elements in a wide range of applications. The microstructures formed by using the nanoparticles are very homogeneous, resulting in site-independent signal intensity.

The functional elements according to the invention may have different microstructures on their carrier surface, the microstructures being made of different nanoparticles with varying molecule-specific recognition sites. Accordingly, these different microstructures may also be assigned various different biofunctions. The functional elements can thus comprise, or be provided with, microstructures with different biological molecules adjacent to each other. A functional element can therefore comprise a plurality of different proteins or a plurality of different nucleic acids, or it may comprise proteins and nucleic acids at the same time.

The functional elements according to the invention can be easily produced using known methods. For example, using suitable suspending agents, stable suspensions can be easily produced from nanoparticles. Nanoparticle suspensions act like solutions and are therefore compatible with microstructuring methods. As a result, nanoparticle suspensions can be structured and deposited directly on suitable carriers, for example by employing conventional methods such as needle-ring printing, lithographic methods, ink jet methods and/or micro-contact methods, these carriers having previously been treated with a bonding agent for firm adhesion of the nanoparticles. By suitably selecting the bonding agent, the formed microstructure can be configured such that it can be partially or entirely detached from the carrier surface of the functional element at a later time, for example by changing the pH value or the temperature, and so that it can optionally be transferred to the carrier surface of a different functional element.

The functional elements according to the invention may be configured in a variety of forms and can therefore be used in very different fields. For example, the functional elements according to the invention can be biochips, such as gene or protein arrays, which are used in the field of medical analysis or diagnostics. The functional elements according to the invention, however, can also be used as electronics components, for example as molecular circuits, in medical measurement and monitoring technologies or in a biocomputer.

In the context of the present invention, a “functional element” shall mean an element, which, either alone or as a component in a more complex device, which is to say, in connection with further similar or different functional elements, performs at least one defined function. A functional element comprises a plurality of components, which can be made of the same material or a different material. The individual components of a functional element can perform various different functions within one functional element and can contribute to the overall function of the element in varying degrees or in varying manners. In the present invention, a functional element comprises a carrier having a carrier surface, on which defined layers of nanoparticles are three-dimensionally disposed as microstructure(s), wherein the nanoparticles are, or can be, provided with biological functions, these being, for example biological molecules such as nucleic acids, proteins and/or PNA molecules, these nanoparticles being protected, preferably by the inclusion of at least one biomolecule-stabilizing agent, and particularly a protein-stabilizing agent.

“Biomolecule-stabilizing agents”, particularly “protein-stabilizing agents” according to the invention shall be understood as agents that stabilize the three-dimensional structure of proteins, which is to say, the secondary, tertiary and quaternary structures, under drying stress and thus maintain the functionality of the proteins when dry, which is to say, following evaporation of the solvent.

In a preferred embodiment, the protein-stabilizing agent is: a saccharide, particularly sucrose, lactose, glucose, trehalose or maltose; a polyalcohol, particularly inositol, ethylene glycol, glycerol, sorbitol, xylithol, mannitol or 2-methyl-2,4-pentanediol; an amino acid, particularly sodium glutamate, proline, alpha-alanine, beta-alanine, glycine, lysine-HCl or 4-hydroxyproline; a polymer, particularly polyethylene glycol, dextran or polyvinylpyrrolidone; an inorganic salt, particularly sodium sulfate, ammonium sulfate, potassium phosphate, magnesium sulfate or sodium fluoride; an organic salt, particularly sodium acetate, sodium polyethylene, sodium caprylate, propionate, lactate or succinate; or trimethylamine N-oxide, sarcosine, betaine, gamma-aminobutyric acid, octopine, alanopine, strombine, dimethylsulfoxide or ethanol; or a mixture of the aforementioned substances.

A “carrier” shall be understood as that component of the functional element, which principally determines the volume and the external shape of the functional element. The term “carrier” particularly refers to a solid matrix. The carrier may have any arbitrary size and any arbitrary shape, for example that of a sphere, a cylinder, a rod, a wire, a plate or a foil. The carrier may be a hollow body or a solid body. A solid body particularly refers to a body that has substantially no cavities and can be made entirely of one material or a combination of materials. The solid body may also be made of a sequence of layers of the same material or different materials.

According to the invention, the carrier of the functional element, and particularly the carrier surface, is made of metal, metal oxide, polymer, glass, semi-conductor material or ceramic material. In the context of the invention, this means that either the carrier is made entirely of one of the above materials, or substantially comprises such a material, or is made entirely of a combination of these materials, or substantially comprises such a combination, or that the surface of the carrier is made entirely of any one of the above materials, or substantially comprises the same, or is made entirely of a combination of these materials, or substantially comprises such a combination. The carrier, or the surface thereof, comprises at least approximately 60%, preferably approximately 70%, more preferred approximately 80% and most preferred approximately 100%, of any one of the above materials, or a combination of such materials.

In a preferred embodiment, the carrier of the functional element is made of materials such as transparent glass, silicon dioxide, metals, metal oxides, polymers and copolymers of dextrans or amides, for example acrylamide derivatives, cellulose, nylon, or polymer materials such as polyethylene terephthalate, cellulose acetate, polystyrene or polymethyl methacrylate, or a polycarbonate of bisphenol A.

The invention provides for the surface of the functional element carrier being planar or pre-structured, for example comprising feed and discharge lines. The invention provides for the surface of the carrier and the carrier itself being impermeable and/or porous. Such carriers are membranes or filters, for example. The invention also provides for the surface sections of the carrier surface that are not covered by the microstructure comprising functionalities and/or chemical compounds, which prevent nonspecific deposition of biomolecules on these surface sections. It is preferable that these chemical compounds be polyethylene glycols, oligoethylene glycols, dextran or a mixture thereof. It is particularly preferred that the surface of the functional element carrier be an ethylene oxide layer.

In a preferred embodiment of the invention, provision is made for at least one layer made of a bonding agent between the carrier surface and the microstructure. The bonding agent serves to ensure tight adhesion of the nanoparticles on the carrier surface of the functional element. The selection of the bonding agent depends on the surface of the carrier material and the nanoparticles to be bound. The bonding agent is preferably a charged or uncharged polymer.

The bonding agents are preferably weak or strong polyelectrolytes, meaning that charge densities thereof are pH-dependent or pH-independent. In a preferred embodiment, the bonding agent comprises poly(diallyl-dimethyl-ammoniumchloride), a sodium salt of poly(styrene sulfonic acid), a sodium salt of poly(vinylsulfonic acid), poly(allylamine-hydrochloride), linear/branched poly(ethylene imine), poly(acrylic acid), poly(methacrylic acid) or a mixture thereof.

The polymer can also be a hydrogel. The bonding agent may be a plasma layer comprising charged groups, such as a polyelectrolyte, or a plasma layer comprising chemically reactive groups. The bonding agent can also be a self-assembled monolayer based on silane, mercaptan, phosphate or fatty acids. The invention provides for the layer of bonding agent comprising at least one layer of a bonding agent. The bonding agent layer, however, may also be made of a plurality of layers comprising different bonding agents, for example an anionic plasma layer and a cationic polymer layer, or of a plurality of polymer layers, which are alternately anionic and cationic.

A further preferred embodiment of the invention relates to bonding agents, the properties of which, such as the cohesion properties thereof, can be varied by an outside stimulus, so that these can be switched externally. For example, the cohesion properties of the bonding agent can be reduced by varying the pH value, the ion concentration and/or the temperature to such an extent that the microstructures bound to the carrier surface of the functional element by the bonding agent can be detached and optionally transferred to the carrier surface of a different functional element.

In a further preferred embodiment of the invention, it is provided that the carrier, and particularly the carrier surface, is pretreated with a surface-activating agent before applying the bonding layers and microstructures, in order to improve the adhesion of the bonding layers and microstructures to be applied to the carrier or the surface thereof. The surfaces of the carrier can be activated, for example, by chemical methods, such as with the use of primers or an acid or a base. Surface activation can also be achieved with the use of plasma. The surface activation step may also comprise the application of a self-assembled monolayer.

A “microstructure” refers to structures in the range of several micrometers or nanometers. Particularly in the context of the present invention, a “microstructure” shall be understood as a structure, which comprises at least two individual components in the form of a plurality of three-dimensionally configured layers of nanoparticles with molecule-specific recognition sites and which is provided on the surface of a carrier, wherein a certain surface section of the carrier surface is covered, this section having a defined shape and a defined surface area and being smaller than the carrier surface. According to the invention, it is particularly provided that at least one of the area-to-length parameters, which define the surface area section covered by the microstructure, is in the micrometer range. If the microstructure has the shape of a circle, for example, the diameter of the circle is in the micrometer range. If the microstructure is configured as a rectangle, the width of this rectangle, for example, is in the micrometer range. The invention particularly provides for at least one of the area-to-length parameters, which define the surface area section covered by the microstructure, being smaller than 999 μm. Since the microstructure according to the invention comprises at least two nanoparticles, the lower limit of this area-to-length parameter is 10 nm.

In a preferred embodiment, the three-dimensionally configured layers of nanoparticles, which collectively form the microstructure, have a total thickness of 10 nanometers to 10 micrometers. According to the invention, a thickness of 50 nanometers to 2.5 micrometers, and particularly a thickness of 100 nanometers to 1.5 micrometers, is preferred.

In the context of the present invention, in contrast to a two-dimensional carrier, which binds capture and/or analyte molecules on a planar surface of an optionally microstructured monolayer, a “three-dimensional” carrier is a carrier, which provides the surface of an optionally microstructured nanoparticle accumulation for the binding of functional groups or biomolecules, wherein an accumulation means two, three or many layers of nanoparticles, which are disposed on top of each other. In particular, the explicit expansion into the third dimension resulting from the described application of optionally microstructured nanoparticle multilayers increases the reactive surface per assigned base area.

The surface area section covered by the microstructure according to the invention may have any arbitrary geometric shape, for example that of a circle, an ellipse, a square, a rectangle or a line. The microstructure, however, may also comprise a plurality of regular and/or irregular geometric shapes. If the functional element according to the invention is a gene chip or a protein array, for example, the microstructure preferably has a circular- or ellipse-like shape. If the functional element according to the invention is an electronics component for use in a biocomputer, the microstructure can have a circuit-like shape. The invention also provides for disposing a plurality of microstructures having the same shape or different shapes at a distance to each other on the carrier surface of a functional element.

The invention provides for the microstructures being applied to the surface of the functional element carrier, for example using one needle-ring printer per ring/pin, by means of lithographic methods, such as photolithography or micro-pen lithography, ink jet techniques or micro-contact printing methods. The selection of the method, which is used to apply the microstructure or microstructures to the surface of the functional element, depends on the surface of the carrier material, the nanoparticles that are intended to form the microstructure and the subsequent field of application of the functional element.

In the context of the present invention, a “nanoparticle” shall be understood as a particulate binding matrix with molecule-specific recognition sites comprising first functional chemical groups. The nanoparticles used according to the invention comprise a core with a surface, on which the first functional groups are disposed, which are able to covalently or non-covalently bind complementary second functional groups of a biomolecule. As a result of interaction between the first and second functional groups, the biomolecule is immobilized on the nanoparticle, and hence on the microstructure of the functional element, and/or can be immobilized thereon. The nanoparticles used according to the invention for forming the microstructures measure less than 500 nm, and preferably less than 150 nm, in size.

In the context of the present invention, “addressable” shall mean that the microstructure can be recovered and/or detected after applying the nanoparticles to the carrier surface. If the microstructure is applied to the carrier surface, for example, with the use of a mask or a stamp, the address of the microstructure is the result of the coordinates x and y of the region of the carrier surface defined by the mask or the stamp, to which region the microstructure is applied. Furthermore, the address of the microstructure results from the molecule-specific recognition sites on the surface of the nanoparticles, which allow for recovery or detection of the microstructure. If the microstructure can be biofunctionalized, which is to say that it comprises nanoparticles with molecule-specific recognition sites to which no biomolecules are bound, the microstructure can be recovered and/or detected in that one or more biomolecules bind specifically to the molecule-specific recognition sites of the nanoparticles forming the microstructure, but not however to the surface area sections of the carrier surface that are not covered by the microstructure. If the immobilized molecule has been labeled, for example, with detection markers such as fluorophenes, spin labels, gold particles, radioactive markers etc., the detection of the microstructure can be carried out by way of suitable detection methods. If the microstructure is biofunctionalized, which is to say it comprises nanoparticles on the molecule-specific recognition sites of which one or more biomolecules have already been bound, “addressable” shall be understood such that these biomolecules can be discovered and/or detected by interaction with the complementary structures of further molecules or by means of metrological methods, wherein only the microstructure comprising the nanoparticles gives off corresponding signals, and the surface area sections of the carrier surface that are not covered by the microstructure do not. Possible detection methods are, for example, matrix assisted laser desorption/ionization time-of-flight mass spectrometry (MALDI-TOF-MS), which has developed into an important method for the analysis of a wide variety of substances, and particularly proteins. Further detection methods are waveguide spectroscopy, fluorescence, impedance spectroscopy, and radiometric and electrical methods.

The invention provides for the biological molecule being bound to or immobilized on, or being capable of being bound to or immobilized on, the surfaces of the nanoparticles forming the microstructure, while preserving biological activity thereof, the molecule preferably being bound at a defined orientation. The biological activity of a molecule refers to all functions that the molecule performs in an organism in the natural cellular environment thereof. If the molecule is a protein, these can be specific catalytic or enzymatic functions, immune defense functions, transport and storage functions, regulating functions, transcription and translation functions and the like. If the molecule is a nucleic acid, the biological function may be, for example, the coding of a gene product or the use of the nucleic acid as a matrix for the synthesis of further nucleic acid molecules or as a binding motif for regulatory proteins. “Preserving the biological activity” shall mean that, following immobilization, a biological molecule can perform the same or nearly the same biological functions on the surface of a nanoparticle, at least to a similar degree, as the same molecule does in the non-immobilized state under suitable in-vitro conditions, or the same molecule in the natural cellular environment thereof.

In the context of the present invention, the term “immobilized in a defined orientation” or “orientated immobilization” shall mean that a molecule is bound at defined positions within the molecule to the molecule-specific recognition sequences of a nanoparticle so that, for example, the three-dimensional structure of the domain(s) required for biological activity is not changed compared with the non-immobilized state, and so that this domain (these domains), for example binding sites for cellular reactants, is (are) freely accessible to these reactants upon contact with other native cellular reactants.

“Immobilized in a defined orientation” shall also mean that during the immobilization of one type of molecule all, or nearly all, individual molecules, and specifically no less than 80%, and preferably more than 85%, of all the molecules reproducibly assume an identical, or nearly identical, orientation on the surface of the nanoparticles forming the microstructure.

The invention provides for the biological molecule that is immobilized, or immobilizable, on the microstructure of the functional element according to the invention being, in particular, a nucleic acid, a protein, a protein complex, a PNA molecule, a fragment thereof or a mixture thereof.

In the context of the present invention, a nucleic acid shall mean a molecule comprising at least two nucleotides that are joined by a phosphodiester bond. The nucleic acid can be a deoxyribonucleic acid or a ribonucleic acid. The nucleic acid may be present in a single-stranded or double-stranded form. In the context of the present invention, a nucleic acid may also be an oligonucleotide. The nucleic acid bound to the microstructure of the functional element according to the invention preferably has a length of at least 10 bases. The nucleic acid may be of natural or synthetic origin. The nucleic acid may be modified by genetic engineering with respect to the wild type nucleic acid and/or may comprise non-natural and/or unusual nucleic acid building blocks. The nucleic acid may be joined with molecules of a different type, for example with proteins.

In the context of the present invention, a “protein” shall mean a molecule comprising at least two amino acids that are joined to each other by an amide bond. In the context of the present invention, a protein can thus also be a peptide, for example an oligopeptide, a polypeptide or, for example, a protein domain. A protein of this type may be of natural or synthetic origin. The protein may be modified by genetic engineering with respect to the wild type protein and/or may comprise non-natural and/or unusual amino acids. Compared with the wild type form, the protein may be derivatized, for example, it may comprise glycosylations, it may be shortened, it may be fused with other proteins or may be joined with molecules of a different type, for example with carbohydrates. According to the invention, a protein can, in particular, be an enzyme, a receptor, a cytokine, a structural protein, an antigen or an antibody.

“Antibody” shall mean a polypeptide, which is substantially coded for by one or more immunoglobulin genes, or fragments thereof, which specifically bind(s) and detect(s) an analyte (antigen). Antibodies occur, for example, as intact immunoglobulins or as a series of fragments, which can be produced by cleavage using different types of peptidases. “Antibodies” shall also mean modified antibodies (for example oligomeric, reduced, oxidized and labeled antibodies). “Antibodies” shall also comprise antibody fragments, which have been produced either by the modification of entire antibodies or by means of de novo synthesis using DNA recombination techniques. The term “antibody” shall encompass both intact molecules and fragments thereof, such as Fab, F(ab′)₂ and Fv, which can bind the epitope determinant.

According to the invention, “protein complex” shall mean a unit of at least two proteins belonging together. In addition to proteins, a “protein complex” may furthermore also comprise nucleic acids, metal ions and other substances.

PNA (Peptide Nucleic Acid or Polyamide Nucleic Acid) molecules are molecules, which are not negatively charged and act in the same manner as DNA (Nielsen et al., 1991, Science, 254, 1497-1500; Nielsen et al., 1997, Biochemistry, 36, 5072-5077; Weiler et al., 1997, Nuc. Acids Res., 25, 2792-2799). PNA sequences comprise a polyamide backbone made of N-(2-aminoethyl)-glycine units and have no glucose units and no phosphate groups.

In the context of the present invention, “molecule-specific recognition sites” shall be understood as regions of the nanoparticles that enable a specific interaction between the nanoparticle and biological molecules that are the target molecules. The interaction may be based on a directed attractive interaction between one or more pairs of first functional groups of the nanoparticle and complementary second functional groups of the target molecules, meaning the biological molecules, the second groups binding the first functional groups. Individual interacting pairs of functional groups between nanoparticle and biological molecule are disposed with spatial fixation on the nanoparticle and the biological molecule. This fixation does not have to be a rigid configuration, but can rather be configured quite flexibly. The attractive interaction between the functional groups of the nanoparticles and the biological molecules can be implemented in the form of non-covalent bonds such as van der Waals bonds, hydrogen bridge bonds, π-π bonds, electrostatic interactions or hydrophobic interactions. Reversible covalent bonds as well as mechanisms that are based on complementariness of shape or form are also conceivable. The interactions provided according to the invention between the molecule-specific recognition sites of the nanoparticles and the target molecule are therefore based on directed interactions between the pairs of functional groups, and on the spatial configuration of these groups that form pairs in relation to each other on the nanoparticle as well as the target molecule. This interaction results in a covalent or non-covalent affinity bond between the two binding partners such that the biological molecule is immobilized on the surface of the nanoparticles forming the microstructure.

In a preferred embodiment of the present invention, the first functional groups, which are part of the molecule-specific recognition sites on the surface of the nanoparticle, or which form the same, are selected from the group consisting of active ester, alkylketone group, aldehyde group, amino group, carboxy group, epoxy group, maleinimido group, hydrazine group, hydrazide group, mercaptan group, thioester group, oligohistidine group, Strep-Tag I, Strep-Tag II, desthiobiotin, biotin, chitin, chitin derivative, chitin-binding domain, metal chelate complex, streptavidin, streptactin, avidin and neutravidin.

The invention also provides for the molecule-specific recognition site being a larger molecule, such as a protein, an antibody or the like, which comprises the first functional groups. The molecule-specific recognition site may also be a molecule complex comprising a plurality of proteins and/or antibodies and/or nucleic acids, wherein at least one of these molecules comprises the first functional groups. A protein may also comprise, for example, an antibody and a protein joined thereto as the molecule-specific detection sequence. The antibody may also comprise a streptavidin group or a biotin group. The protein joined to the antibody can be a receptor, for example an MHC protein, cytokine, a T-cell receptor such as the CD 8 protein and others, wherein the receptor can bind a ligand. A molecule complex may also comprise a plurality of proteins and/or peptides, for example a biotinylated protein, which in a complex binds a further protein as well as a peptide.

The second functional group, which is to say, the functional group of the biomolecule to be immobilized, according to the invention is selected from the group consisting of active ester, alkylketone group, aldehyde group, amino group, carboxy group, epoxy group, maleinimido group, hydrazine group, hydrazide group, mercaptan group, thioester group, oligohistidine group, Strep-Tag I, Strep-Tag II, desthiobiotin, biotin, chitin, chitin derivative, chitin-binding domain, metal chelate complex, streptavidin, streptactin, avidin and neutravidin.

The first and second functional groups can have been produced by molecular imprinting, for example. The first and second functional groups can also be active esters, such as those referred to as surfmers.

A nanoparticle that is used according to the invention on the surface thus has a first functional group, which is associated covalently or non-covalently with a second functional group of a biomolecule to be immobilized, the first functional group being a different group than the second functional group. The two groups forming a bond with each other must be complementary to each other, meaning they must be capable of entering a covalent or non-covalent bond with each other.

If, according to the invention, for example an alkyl ketone group, particularly a methyl ketone or aldehyde group is used as the first functional group, then the second functional group is a hydrazine or hydrazide group. Conversely, if a hydrazine or hydrazide group is used as the first functional group, then according to the invention the second functional group is an alkyl ketone, particularly a methyl ketone or an aldehyde group. If, according to the invention, a mercaptan group is used as the first functional group, then the second complementary functional group is a thioester group. If, according to the invention, a thioester group is used as the first functional group, then the second functional group is a mercaptan group.

If, according to the invention, a metal ion chelate complex is used as the first functional group, then the second functional complementary group is an oligohistidine group. If, according to the invention, an oligohistidine group is used as the first functional group, then the second functional complementary group is a metal ion chelate complex.

If Strep-Tag I, Strep-Tag II, biotin or desthiobiotin is used as the first functional group, then streptavidin, streptactin, avidin or neutravidin is used as the second complementary functional group. If streptavidin, streptactin, avidin or neutravidin is used as the first functional group, then Strep-Tag I, Strep-Tag II, biotin or desthiobiotin is used as the second complementary functional group.

If, in a further embodiment, chitin or a chitin derivative is used as the first functional group, then a chitin-binding domain is used as the second functional complementary group. If a chitin-binding domain is used as the first functional group, then chitin or a chitin derivative is used as the second functional complementary group.

According to the invention, the aforementioned first and/or second functional groups can be joined to the biomolecule to be immobilized, or to the nanoparticle core, with the help of a spacer or can be inserted into the nanoparticle core, or into the biomolecule, with the help of a spacer. The spacer thus serves, on one hand, to keep the functional group at a distance from the core, or from the biomolecule, and, on the other hand, as carrier for the functional group. According to the invention, a spacer of this type may comprise alkylene groups or ethylene oxide oligomers with 2 to 50 carbon atoms and, in a preferred embodiment, is substituted and comprises heteroatoms.

In a preferred embodiment of the invention, it is provided that the second functional groups are natural components of the biomolecule, particularly of a protein.

An average sized protein, meaning a size of approximately 50 kDA with approximately 500 amino acids, has about 20 to 30 reactive amino groups, which in principle are potential functional groups for immobilization purposes. In particular, this will be the amino group on the N-terminal end of a protein. All remaining free amino groups, particularly the lysine radicals, may also be used for the immobilization process. Arginine, which has a guanidium group, is a also a potential functional group.

A further preferred embodiment of the invention provides for introducing the second functional groups in the biomolecule by means of genetic engineering methods, biochemical, enzymatic and/or chemical derivatization, or chemical synthesis methods. The derivatization should be carried out so that biological activity is preserved following immobilization.

If the biomolecule is a protein, it is possible, for example, to insert non-natural amino acids into the protein molecule by means of genetic engineering methods or during chemical protein synthesis, for example in conjunction with spacers or linkers. Such non-natural amino acids are compounds with an amino acid function and a radical R, which are not defined by a naturally occurring genetic code, and in a particularly preferred embodiment these amino acids comprise a mercaptan group. The invention may also provide for modifying a naturally occurring amino acid, such as lysine, for example by means of derivatization of the side chain thereof, particularly the primary amino group thereof, using the carboxylic acid function of levulinic acid.

In a further preferred embodiment of the present invention, functional groups can be inserted in a protein by modification of the same, whereby tags, which is to say markers, are added to the protein, preferably on the C-terminal or the N-terminal. However, these tags may also be provided intramolecularly. In particular, it is provided that a protein is modified in that at least one Strep-Tag, for example Strep-Tag I or Strep-Tag II or biotin, is added. According to the invention, a Strep-Tag shall also be understood as functional and/or structural equivalents thereof, provided that they are able to bind streptavidin groups and/or equivalents thereof. The term “streptavidin” within the meaning of the present invention shall also include functional and/or structural equivalents thereof. The invention also provides for modifying a protein by adding a His tag, comprising at least three histidine groups, and preferably an oligohistidine group. The His tag introduced in the protein may then bind to a molecule-specific recognition site comprising a metal chelate complex.

A preferred embodiment of the invention thus provides for bringing about a binding step for proteins, which are modified, for example, with non-natural amino acids, natural but non-naturally derivatized amino acids or specific Strep tags, or proteins that are bound to antibodies with complementary reactive nanoparticle surfaces, so that suitable specific, and particularly non-covalent, binding of the proteins, and hence directed immobilization of the proteins, occurs on the surfaces. Following the orientation of the bioactive molecules using tag binding sites, these molecules can be further covalently bound, for example using a cross-linker such as glutaraldehyde. As a result, the protein surfaces become more stable.

In addition to the surface having the molecule-specific recognition sites, the nanoparticles precipitated on the carrier surface of the functional element for forming a microstructure also have a core. In the context of the present invention, a “core” of a nanoparticle shall be understood as a chemically inert substance, which serves as carrier for the molecule to be immobilized. According to the invention, the core is a compact or hollow particle measuring between 5 nm and 500 nm in size.

In a preferred embodiment of the present invention, the core of the nanoparticles used according to the invention is made of, or comprises, an inorganic material, such as a metal, for example Au, Ag or Ni, silicon, SiO₂, SiO, a silicate, Al₂O₃, SiO₂·Al₂O₃, Fe₂O₃, Ag₂O, TiO₂, ZrO₂, Zr₂O₃, Ta₂O₅, zeolite, glass, indium tin oxide, hydroxylapatite, a Q-Dot or a mixture thereof.

In a further preferred embodiment of the invention, the core is made of, or comprises, an organic material. It is preferable that the organic polymer be polypropylene, polystyrene, polyacrylate, a polyester of lactic acid or a mixture thereof.

The cores of the nanoparticles used according to the invention can be produced using conventional methods that are known in the field, such as sol-gel synthesis, emulsion polymerization, suspension polymerization etc. After producing the cores, the surfaces of the cores are provided with the specific first functional groups by chemical modification reactions, for example, by conventional methods such as graft polymerization, silanization, chemical derivatization and the like. One possible way of producing surface-modified nanoparticles in a single operation is to use surfmers in the emulsion polymerization process. Another possibility is molecular imprinting.

Molecular imprinting is the polymerization of monomers in the presence of templates, which can form a complex with the monomer, the complex being relatively stable during polymerization. After washing out the templates, the resulting materials can again specifically bind the template molecules, molecules of a type that is structurally related to the template molecules, or molecules having groups that are structurally related to the template molecules, or parts thereof, or identical groups. A template is thus a substance, which is present in the monomer mixture during polymerization, and for which the formed polymer exhibits an affinity.

According to the invention, it is particularly preferred to produce surface-modified nanoparticles by means of emulsion polymerization, using surfmers. Surfmers are amphiphilic monomers (surfmer=surfactant+monomer), which can be polymerized on the surface of latex particles, and which stabilize the same. Reactive surfmers additionally comprise functionalizable end groups, which can be reacted under mild conditions with nucleophiles, such as primary amines (amino acids, peptides, proteins), mercaptan or alcohols. In this way, a variety of biologically active polymer nanoparticles are accessible. Published prior art that reflects the state of the art as well as possibilities and limits for the application of surfmers includes U.S. Pat. No. 5,177,165, U.S. Pat. No. 5,525,691, U.S. Pat. No. 5,162,475, U.S. Pat. No. 5,827,927 and JP 4 018 929. Papers relating to the synthesis of surfmers using reactive terminal groups include publications by Nagai et al. (Polymer 1996, 37(1), 1257-1266; Journal of Colloid and Interface Science 1995, 172, 63-70), Asua et al. (J. Applied Polym. Sci. 1997, 66, 1803-1820) and Guyot et al. (Curr. Opin. Colloid Interface Sci. 1996, 1(5), 580-586).

According to the invention, the density of the first functional groups and the distance of these groups to each other can be optimized for each molecule that is to be immobilized. Also the environment of the first functional groups on the surface can be prepared accordingly in terms of the best possible specific immobilization of a Biomolecule.

A preferred embodiment of the invention provides for additional functions being anchored in the core, these functions allowing for ready detection of the nanoparticle cores, and thus of the microstructures, through the use of suitable detection methods. These functions may be, for example, fluorescence markers, UV-Vis markers, superparamagnetic functions, ferromagnetic functions and/or radioactive markers. Suitable methods for detecting nanoparticles include, for example, fluorescence or UV-Vis spectroscopy, fluorescence or light microscopy, MALDI mass spectrometry, waveguide spectroscopy, impedance spectroscopy, electrical and radiometry methods. A combination of the methods may also be used for detecting the nanoparticles. A further embodiment provides that the core surface can be modified by applying further functions such as fluorescence markers, UV-Vis markers, superparamagnetic functions, ferromagnetic functions and/or radioactive markers. A further embodiment of the invention provides that the core of the nanoparticle can be surface-modified with an organic or inorganic layer comprising the first functional groups and the aforementioned additional functions.

A further embodiment of the invention provides for the core surface comprising chemical compounds, which serve to provide steric stabilization and/or to prevent conformation changes in the immobilized molecules and/or to prevent deposition of further biologically active compounds on the core surface. It is preferable that these chemical compounds be polyethylene glycols, oligoethylene glycols, dextran or a mixture thereof.

According to the invention, it is also possible to separately or additionally anchor ion exchange functions to the surface of the nanoparticle cores. Nanoparticles with ion exchange functions are particularly suited for optimizing MALDI analysis because this allows interfering ions to be bound.

A further embodiment of the invention provides for the biological molecule that is immobilized on the microstructure of the functional element having markers that enable ready detection of the biological molecule that is immobilized on the microstructure with the use of suitable detection methods. These markers can be, for example, a fluorescence marker, a UV-V is marker, a superparamagnetic function, a ferromagnetic function and/or a radioactive marker. As described above, possible detection methods for these markers are, for example, fluorescence or UV-Vis spectroscopy, MALDI mass spectrometry, waveguide spectroscopy, impedance spectroscopy, electrical and radiometry methods or a combination of these methods.

A further embodiment of the invention relates to a functional element having at least one biological molecule immobilized on the microstructure, wherein at least one further biological molecule is covalently or non-covalently bound to this immobilized molecule. If the molecule immobilized on the microstructure is a protein, for example, a second protein or an antibody can be bound thereto, for example by protein/protein interaction or by antibody/antigen binding. If the molecule immobilized on the microstructure is a nucleic acid, a protein may be bound thereto, for example.

A further preferred embodiment of the invention relates to a functional element, the microstructure(s) of which comprise(s) a plurality of layers of the same nanoparticles, these layers being disposed on top of each other, wherein each individual layer is bound to the layer underneath by the bonding layers described above, which are made of suitable polymers.

A further preferred embodiment of the invention relates to a functional element, on the carrier surface of which a plurality of different microstructures are adjacently disposed, which are made of nanoparticles with different molecule-specific recognition sites. Functional elements of this type therefore comprise adjacent microstructures, on which the different biological molecules are, or can be, immobilized. Therefore, the carrier surface of such functional elements can, for example, comprise both microstructures on which proteins are, or can be, immobilized, and microstructures on which nucleic acids are, or can be, immobilized. However, the functional element can also comprise microstructures on which different proteins or different nucleic acids are, or can be, immobilized at the same time.

A further embodiment of the invention relates to a functional element, on which the surface sections of the carrier surface that are not covered by the microstructure are modified, for example by applying further functionalities and/or chemical compounds. These can, in particular, be functionalities and/or chemical compounds, which prevent nonspecific deposition of biomolecules on the regions of the carrier surface that are not covered by the microstructure. It is preferable that these chemical compounds be polyethylene glycols, oligoethylene glycols, dextran or a mixture thereof. It is particularly preferred that the surface of the functional element carrier be an ethylene oxide layer.

The present invention also relates to a method for producing a functional test according to the invention, at least one layer of a bonding agent being applied to the surface of a suitable carrier and thereafter at least one microstructure comprising nanoparticles with molecule-specific recognition sequences.

The invention provides for the surface of a functional element being pre-structured before applying the layer of the bonding agent. After pre-structuring the carrier surface, a layer of a compound can be applied to the pre-structured carrier surface, this compound preventing a nonspecific deposition of biological molecules on the carrier surface. This is preferably an ethylene oxide layer.

A preferred embodiment of the invention provides for the surface of the carrier of the functional element according to the invention being activated after pre-structuring and before applying the layer of the bonding agent. According to the invention, the activation process may also include purification. According to the invention, the surface of the carrier of the functional element can be activated by using a chemical process, particularly through the use of primers or acids or bases. According to the invention, however, it is also possible to activate the surface of the carrier through the use of plasma. The activation step may also comprise the application of a self-assembled monolayer.

To produce the microstructures on the carrier surface of the functional element in accordance with the invention, in principle, the following two embodiments may be used.

The first embodiment of the method according to the invention for producing a functional element according to the invention provides for first applying the bonding agent in a structured manner to the surface of the carrier. “Structured” in the context of the invention shall mean that a bonding agent layer is applied to the carrier surface, this bonding agent layer being defined in terms of shape and surface area, so that the bonding agent layer applied this way defines the surface area section of the carrier surface to be subsequently covered by the microstructure. The microstructure is then applied by immersing the functional element carrier in a nanoparticle suspension, whereby the nanoparticles adhere only to the structured applied bonding agent layer, but not to the surface area sections of the carrier surface to which no bonding agent layer has been applied. In this way, a microstructure is produced, which is defined in terms of shape and surface area.

The net surface charge of functional nanoparticles depends on the surface modification and the suspension medium. A surface charge other than 0 stabilizes the particle suspension (electrostatic repulsion).

It has been found that aqueous solutions are well suited as suspension media for applying nanoparticle layers in a microstructured form on surfaces coated with polyelectrolyte (for example silicon or glass). In this medium, the particles generally have a net negative charge. The zeta potentials of functionalized nanoparticles are, for example:

Carboxy-functionalized nanoparticles in MilliQ H₂O: −45 mV

Rabbit IgG particles in MilliQ H₂O: −40 mV

Rabbit IgG particles in MilliQ H₂O+5% trehalose: −37 mV

Goat IgG particles in MilliQ H₂O: −36 mV

Goat IgG particles in MilliQ H₂O+5% trehalose: −24 mV

SAv particles in MilliQ H₂O+0.5% trehalose: −53 mV

StrepTactin® particles in MilliQ H₂O: −43 mV

Protein G particles in MilliQ H₂O: −49 mV.

The invention provides for the structured bonding agent layer being applied, for example, by means of a needle-ring printer, an ink jet method such as a piezo or thermal method, or a micro-contact printing method. When using a lithographic method, and in particular the photolithography or the micropen lithography method, the carrier surface is covered with the bonding agent and the bonding agent layer produced in this manner is then structured by means of the lithographic method. By suitably selecting the bonding agent, the microstructure to be applied can be configured so that the microstructure, or parts thereof, can be switched externally and subsequently detached again (debond on command), for example by changing the pH value, the ion concentration or the temperature. In this way, for example, a microstructure can be transferred from one functional element to another.

Stable nanoparticle suspensions can be easily produced by suspending the nanoparticles in fluids, and particularly aqueous media, optionally with additional components, such as pH agents, suspending aids and the like.

The second embodiment of the method according to the invention for producing a functional element, provides for first applying to the carrier a bonding agent layer covering the entire carrier surface. This can be done, for example, by immersing the carrier in a suspension or solution of the bonding agent. Thereafter, the microstructure is produced by applying a nanoparticle suspension in a structured manner, for example with the use of a needle-ring printer, an ink jet method such as a piezo or thermal method, or a micro-contact printing method, so as to produce a microstructure, which is defined in terms of shape and surface area. When using a lithographic method, and particularly the photolithography or the micropen lithography method, the carrier surface is covered with the nanoparticle suspension and the nanoparticle layer produced this way is then structured by means of the lithographic method.

The nanoparticles, which are applied to the carrier surface of a functional element according to the invention for the purpose of producing microstructures, can be biofunctionalizable nanoparticles, meaning nanoparticles, which comprise exclusively molecule-specific recognition sites, and to which no biological molecules are yet bound. According to the invention, however, it is also possible to use biofunctionalized nanoparticles for structuring the carrier surface, which is to say nanoparticles, on the molecule-specific recognition sites of which biological molecules have already been immobilized, while preserving the biological activity thereof. The invention provides for the immobilized biological molecule being, in particular, a protein, a PNA molecule or a nucleic acid.

In a further preferred embodiment of the invention, provision is made for repeated applications of the same bonding agent and/or the same nanoparticles in order to produce multi-layer microstructures with firm adhesion. It is possible, according to the invention, to repeat any one of the aforementioned methods up to ten times. The methods described above, however, can also be repeated using different bonding agents and/or different nanoparticles in order to produce functional elements with different microstructures having different functions.

A further preferred embodiment of the invention provides for disposing superparamagnetic or ferromagnetic iron oxide nanoparticles on a carrier surface in a structured manner, using a magnetic field, and in this way directly build microstructures, particularly nanoscopic printed circuit board tracks.

After applying the nanoparticles to the carrier surface of the functional element, it is possible, according to the invention, to convert the particles further. If the particles comprise reactive esters, for example, then these can be used for the direct binding of proteins. The nanoparticles, however, can also be converted to provide further functions. According to the invention, it is also possible to additionally fix the microstructures made of nanoparticles in place by covalently cross-linking the particles to each other, and/or with the binding agent, for example.

The present invention also relates to the use of the functional element according to the invention for analyzing an analyte in a sample and/or for isolating the same and/or for purifying the same, wherein the functional element according to the invention is a gene array or gene chip or protein array, for example. In the context of the present invention, an “analyte” shall be understood as a substance, the type and quantity of the individual components of which are to be determined and/or which is to be separated from mixtures. In particular, the analyte is a protein, nucleic acid, a carbohydrate or the like. In a preferred embodiment of the invention, the analyte is a protein, peptide, active ingredient, harmful substance, toxin, pesticide, antigen or nucleic acid. A “sample” shall mean an aqueous or organic solution, emulsion, dispersion or suspension, which comprises one of the analytes defined above in isolated and purified form or as a component of a complex mixture of different substances. A sample can be, for example, a biological fluid such as blood, lymph fluid, tissue fluid, or the like, which is to say, a fluid that was removed from a living or dead organism, organ or tissue. A sample, however, may also be a nutrient solution, for example a fermentation medium, in which organisms, for example microorganisms, or human, animal or plant cells have been cultivated. A sample as defined by the invention, however, can also be an aqueous solution, emulsion, dispersion or suspension of an isolated and purified analyte. A sample can already have undergone purification steps, or it may be present in the unpurified state.

The present invention therefore also relates to the use of the functional element according to the invention for carrying out analytical and/or detection methods, these methods being MALDI mass spectrometry, fluorescence or UV-Vis spectroscopy, fluorescence or light microscopy, waveguide spectroscopy, an electrical method such as impedance spectroscopy, or a combination of these methods.

The present invention also relates to the use of a functional element according to the invention for controlling cell adhesion or cell growth.

The present invention furthermore relates to the use of a functional element according to the invention for detecting and/or isolating biological molecules. For example, a functional element according to the invention, on the microstructures of which a preferably single-stranded nucleic acid is immobilized, can be used for detecting a complementary nucleic acid in a sample and/or for isolating this complementary nucleic acid. A functional element according to the invention, on the microstructures of which a protein is immobilized, can be used, for example, for the detection and/or isolation of a protein interacting with the immobilized protein from a sample.

The present invention further relates to the use of a functional element according to the invention for the development of pharmaceutical preparations. The invention also relates to the use of the functional element according to the invention for analyzing the effects and/or side effects of pharmaceutical preparations. The functional elements according to the invention can likewise be used for diagnosing diseases, for example for identifying pathogens and for identifying mutated genes that result in diseases. A further possible application of the functional elements according to the invention is the analysis of microbiological contaminations of surface water, ground water and soil. The functional elements according to the invention can likewise be used for the analysis of the microbiological contamination of foods and/or animal feed.

A further preferred application of the functional elements according to the invention is the use of the functional element according to the invention as an electronics component, for example as a molecular circuit or the like, in medical technology or in a biocomputer. The use of the functional element according to the invention as optical storage media in optical data processing is particularly preferred, in particular wherein the functional element according to the invention comprises photoreceptor proteins immobilized on microstructures, these proteins being able to convert light directly into a signal.

The present invention further relates to a method for identifying and/or detecting analytes in a solution or a suspension. According to the method, in a first step, a functional element according to the invention is provided, which, in a second step, is brought in contact with a solution or suspension comprising an analyte. In a third step, non-bound analyte is removed from the functional element according to the invention by washing it with a biocompatible washing fluid. In a subsequent fourth step, a detection procedure is carried out.

In a preferred method according to the invention, the biocompatible washing fluid is water and/or buffer, for example phosphate-buffered saline: PBS, and/or buffer with an added detergent, for example TritonX-100. In a preferred embodiment of the invention, the carrier can be washed at room temperature sequentially in water and buffer, optionally using a detergent, or in buffer, optionally using a detergent, and water, for example for 30 minutes, respectively.

In a preferred embodiment of the method, a fluorescence detection method or a MALDI mass spectrometry method is used as the detection method for the analyte in a solution or suspension.

Therefore, according to the invention, a method of the sort described above is particularly preferred for the identification and/or for the detection of analyte in a solution or suspension, wherein following the completion of the first three steps, in a fourth step, according to which, in a preferred embodiment, a fluorescence detection method is used, a fluorescence-labeled analyte and/or fluorescence-labeled biologically active molecule that is bound to the nanoparticle is excited with light and read with light.

It is preferred, according to the invention, that the analyte and/or the biological molecule that is bound to the molecule-specific recognition sites of the nanoparticles be fluorescence-labeled.

Further advantageous embodiments of the invention will be apparent from the dependent claims.

The invention will be explained in more detail hereinafter with reference to the figures and examples provided below.

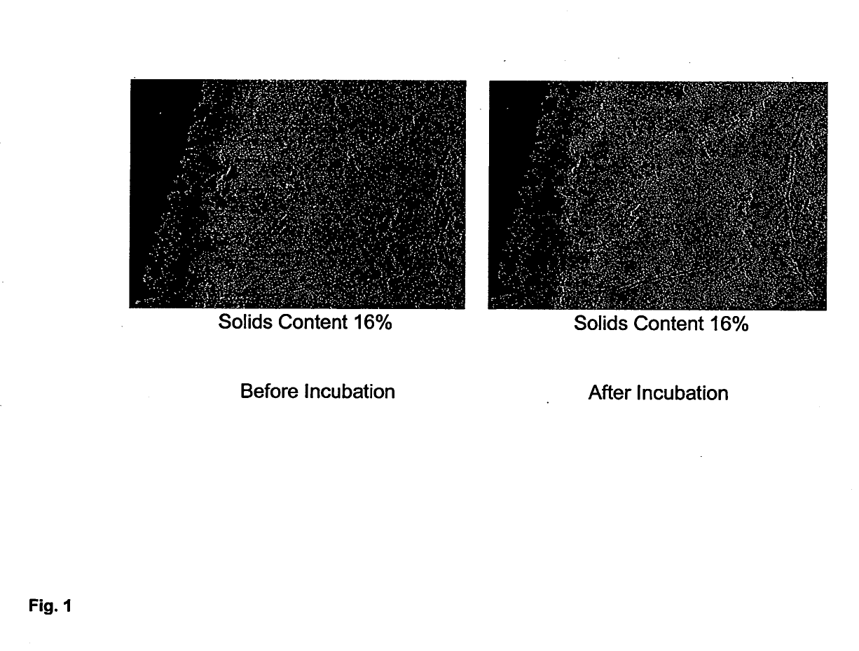

FIG. 1 shows scanning electron microscope images of a three-dimensional nanoparticle structure (section of a microspot, diameter ˜150 μm), produced by quintuple application of a 16% nanoparticle suspension in an aqueous trehalose solution (5% w/v).

Left: The nanoparticulate 3D structure immediately after spotting the nanoparticles on a silicon carrier coated with polyelectrolyte. Right: The same structure after 2 hours of incubation in PBS buffer and 30 minutes of washing in each of PBS/0.1% TritonX-100, PBS and MilliQ water.

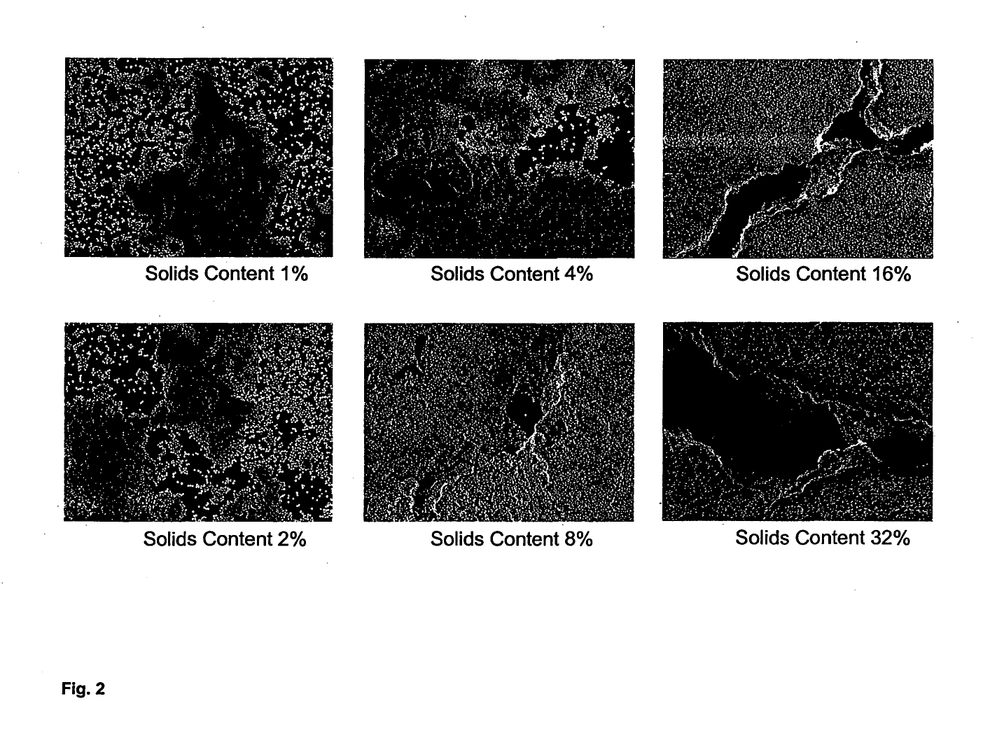

FIG. 2 shows scanning electron microscope images of nanoparticle layers (microspot sections, diameter ˜150 μm), produced by quintuple application of nanoparticle suspension (1%, 2%, 4%, 8%, 16%, and 32%) in aqueous trehalose solution (5% (w/v)) on silicon surfaces coated with polyelectrolyte. The particle surfaces were incubated for 2 hours in PBS buffer and washed 30 minutes each in PBS/0.1% TritonX-100, PBS and MilliQ water.

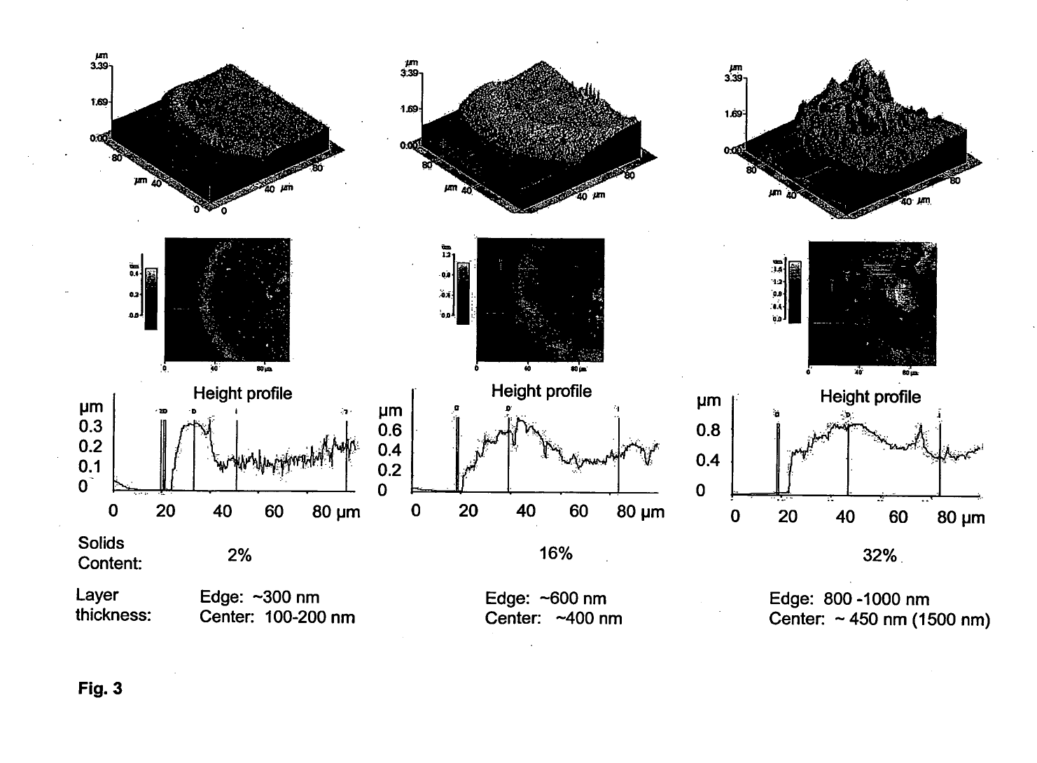

FIG. 3 shows atomic force microscope images (100×100 μm²) of three-dimensional nanoparticle microstructures (microspot sections, diameter ˜150 μm), produced by quintuple application of 2%, 16% and 32% nanoparticle suspension in an aqueous trehalose solution (5% (w/v)) on silicon surfaces coated with polyelectrolyte. 3D illustration, gray scale illustration, height profile along the line marked in the gray scale illustration.

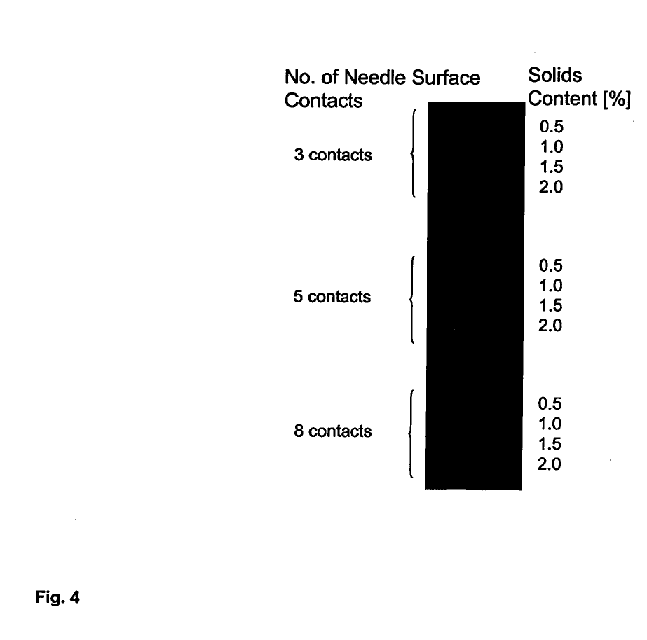

FIG. 4 shows the fluorescence scan of a nanoparticle microarray. Spotting was carried out with Rabbit IgG nanoparticles, the incubation was carried out with Cy5-labeled anti-rabbit IgG. The bound analyte quantity per spot increases with the number of particles that are transferred per spot.

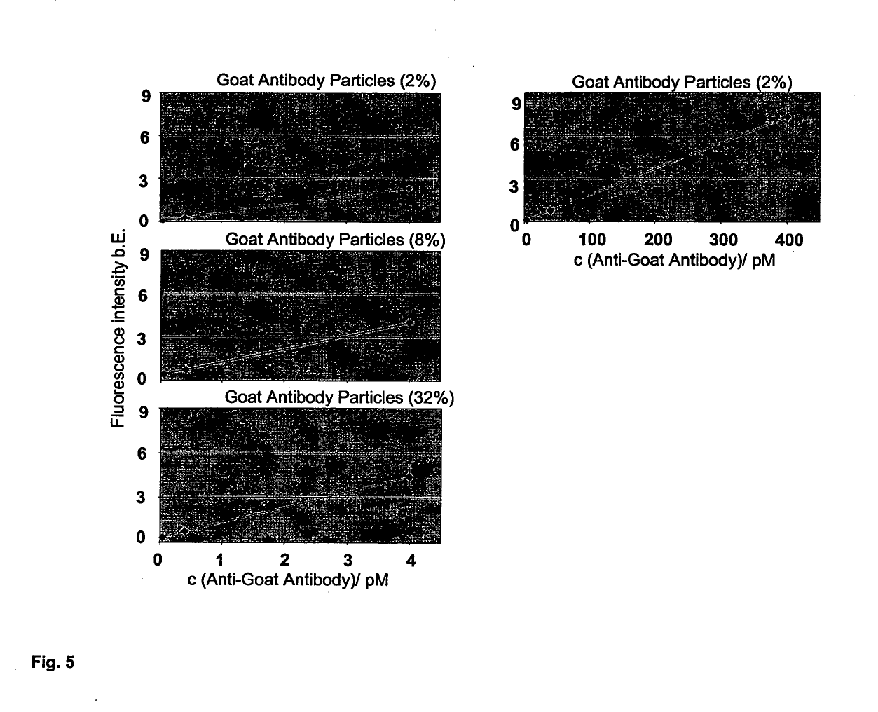

FIG. 5 shows the linear correlation between signal intensity and analyte concentration. Spotting was carried out with Goat IgG and Rabbit IgG nanoparticles. Different concentrations of Cy5-labeled anti-goat and Cy3-labeled anti-rabbit IgG were used for incubation. Sample concentrations on the left: 0.04-4 pM, on the right: 4-400 pM. Left: The sensitivity (incline) increases with larger particle amounts per spot.

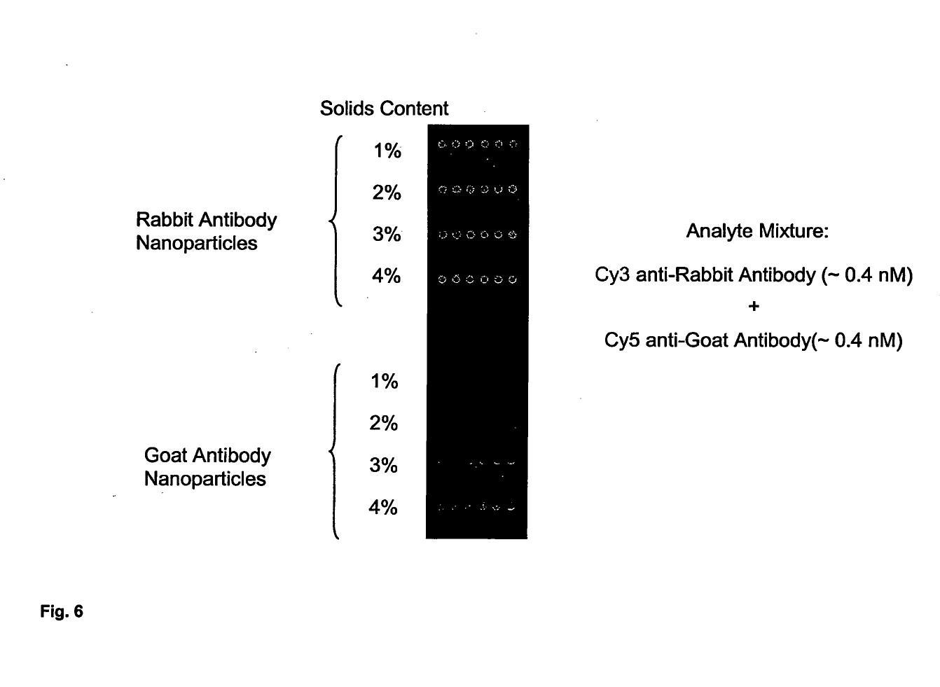

FIG. 6 shows the fluorescence scan of a nanoparticle microarray. Spotting was carried out with Goat IgG and Rabbit IgG nanoparticles in suspensions with different solids contents. Solutions of Cy5-labeled anti-goat and Cy3-labeled anti-rabbit IgG were used for incubation. The nanoparticulate microstructures only bind selectively to the specific interaction partners of the immobilized capture molecules.

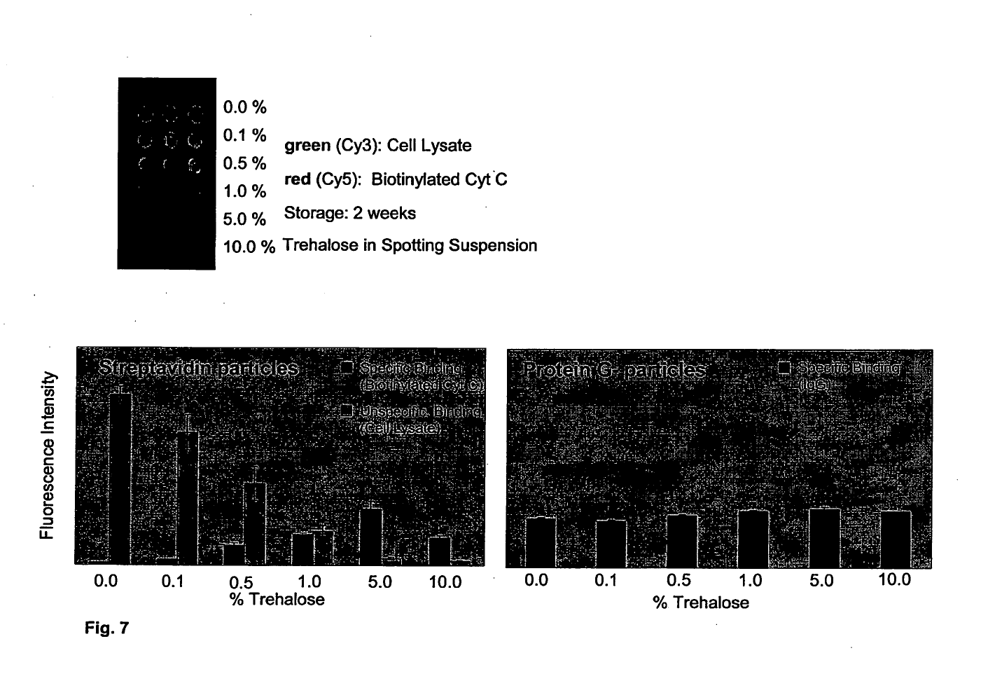

FIG. 7 shows the dependency of the preservation of the function of particle-bound capture proteins on the trehalose concentration in the spotting suspension.

Top: Fluorescence scan of a nanoparticle microarray: Spotting was carried out with streptavidin nanoparticles. The incubation was carried out after 2 weeks of storage with biotinylated, Alexa647-labeled cytochrome C and Alexa546-labeled cell lysate (not biotinylated).

Bottom: Fluorescence intensity as a function of the trehalose content of the spotting suspension: Spotting was carried out with streptavidin nanoparticles and G protein nanoparticles. The incubation was carried out after 2 weeks of storage with biotinylated, Alexa647-labeled cytochrome C and Alexa546-labeled cell lysate (not biotinylated) and FITC-labeled Mouse IgG.

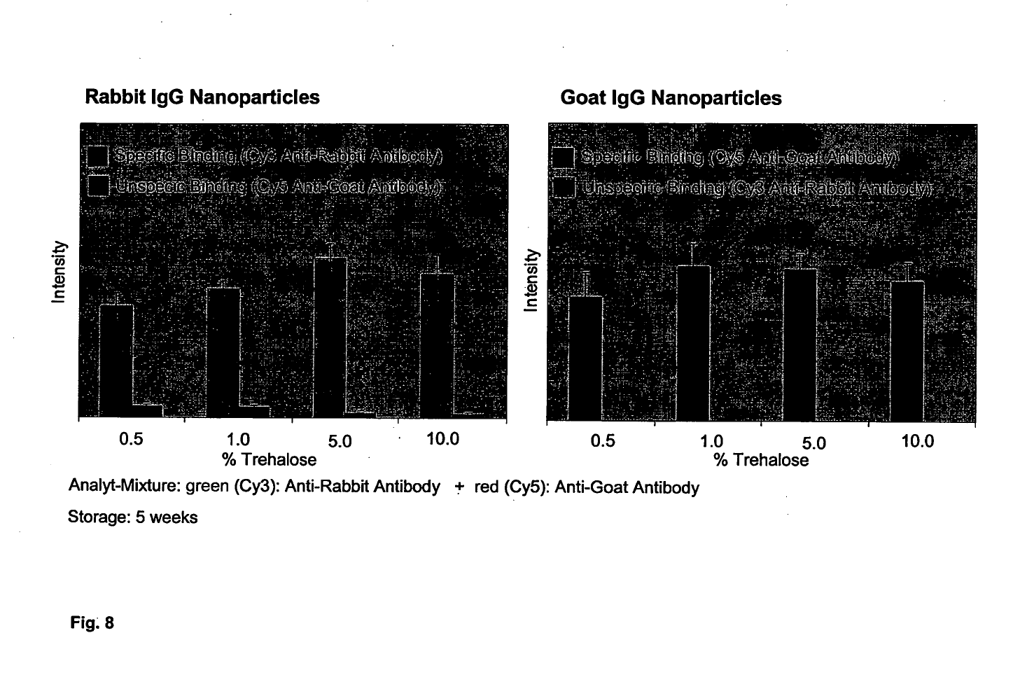

FIG. 8 shows the dependency of the preservation of the function of particle-bound capture proteins on the trehalose concentration in the spotting suspension. Fluorescence intensity as a function of the trehalose content of the spotting suspension: Spotting was carried out with Goat IgG nanoparticles and Rabbit IgG nanoparticles. Both chip types were incubated after 5 weeks of storage with Cy5-labeled anti-goat IgG and Cy3-labeled anti-rabbit IgG.

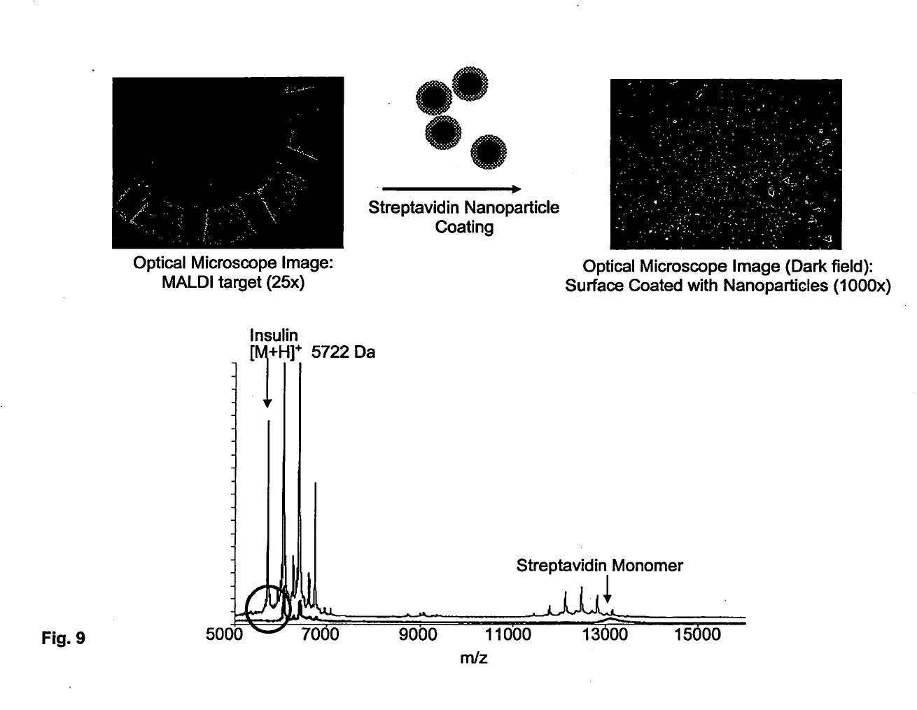

FIG. 9 shows an optical microscope image of a gold substrate before the immobilization of nanoparticles thereon (25×) and an image of the surface coated with streptavidin particles (1000×, dark field). The upper spectrum (blue) is a MALDI TOF spectrum of the analyte solution, which was used to incubate the nanoparticle surface (biotinylated and non-biotinylated insulin). The bottom spectrum is the MALDI TOF spectrum of the nanoparticle-bound molecules after incubation.

### EXAMPLE 1

### Production of Nanoparticle-Based Protein Biochips for Fluorescence Readout

Substrate:

Glass substrates are used for the production of nanoparticle-based protein biochips that are suited for fluorescence readout. Adhesion of the nanoparticles to the surfaces is primarily provided by electrostatic interaction. For the adsorption of protein-coated nanoparticles on the substrate, generally positively charged surfaces are required. Commercially available glass microscope slides with positive groups on the surface are imprinted with protein-coated nanoparticles without further pretreatment.

Conventional glass surfaces are cleaned in a 2% by volume aqueous HELLMANEX® solution for 90 minutes at 40° C. After washing them in MilliQ-H₂O (deionized water, 18 MΩ), the glass slides are hydroxylated in a 3:1 (v/v) NH₃/H₂O₂ solution for 40 minutes at 70° C. (NH₃: puriss. p.a. ˜25% in water, H₂O₂: for analysis, ISO reagent, stabilized).

Following thorough washing in MilliQ-H₂O, the substrates are incubated at room temperature for 20 minutes in an aqueous polycation solution (0.02 M poly(allylamine) (in relation to the monomer), pH 8.5), washed for 5 minutes in MilliQ-H₂O and then centrifuged until dry.

Synthesis of Silica Particles:

Tetraethoxysilane in the amount of 12 mmol and 90 mmol of NH₃ are added to 200 ml of ethanol. The mixture is stirred at room temperature for 24 hours. Thereafter, the particles are purified by multiple centrifugation. This results in 650 mg of silica particles with a mean particle size of 125 nm.

Amino-Functionalization of Silica Particles:

A 1% by weight aqueous suspension of the silica particles is mixed with 10% by volume of 25% ammonia. Thereafter, 20% by weight of aminopropyl triethoxy silane, in relation to the particles, is added and the mixture is stirred for 1 hour at room temperature. The particles are purified by multiple centrifugation and carry functional amino groups on the surfaces thereof (zeta potential in 0.1 M acetate buffer: +35 mV).

Carboxy-Functionalization of Silica Particles:

A 2% by weight suspension of amino-functional nanoparticles in the amount of 10 ml is placed in tetrahydrofuran. To this, 260 mg of succinic acid anhydride is added. Following a 5-minute ultrasound treatment, the mixture is stirred for 1 hour at room temperature. The particles are washed by repeat centrifugation and carry functional amino groups on the surfaces thereof (zeta potential in 0.1 M acetate buffer: −35 mV). The mean particle size is 170 nm.

IgG Binding to Silica Particles:

Carboxy-functionalized silica particles in the amount of 1 mg are mixed with 4 μl of an IgG solution (11.3 mg/ml) und 10 μL of an EDC solution (N-(3-dimethylaminopropyl)-N′-ethylcarbodiimide hydrochloride; 3.8 mg/ml) and brought to 1 ml with MES buffer (pH 4.5).

The mixture is shaken for 3 hours at room temperature and then the particles are washed by repeated centrifugation (zeta potential of IgG particles in MilliQ water: −40 mN).

Preservation of Protein Function in Nanoparticle Layers:

In order to stabilize the function of nanoparticle-bound capture proteins in nanoparticle layers, the particles are suspended for coating in a 5% (w/v) aqueous trehalose solution (FIGS. 7, 8).

Production of Nanoparticle Protein Microarrays:

For the production of fluorescence-readable IgG nanoparticle chips, rabbit and/or goat IgG-coated nanoparticles are transferred to the pretreated glass substrate with the help of a pin ring spotter. The concentration of the particle suspensions used is 0.5% to 50% (w/v) (FIGS. 1-6). Each pin contact with the surface transmits approximately 50 pl of suspension; the printing step is repeated five times per spot. The resulting nanoparticle layers are 100 nm to 1500 nm thick. The spot diameter is approximately 150 μm. The placement of the individual spots on the substrate is freely programmable.

Use of Nanoparticle Protein Biochips:

First, the nanoparticle surfaces are blocked for one hour with a 3% (w/v) solution of BSA in PBS buffer, then incubated for 1.5 hours in the dark at room temperature with the analyte solution, and thereafter washed for 30 minutes each in PBS/0.1% TritonX 100, PBS and MilliQ water. All the steps are carried out in glass microscope slide stands.

The analyte solution comprises the fluorescence-labeled IgG molecules dissolved in PBS buffer (FIG. 4: 40 pM Cy5-labeled anti-rabbit IgG, FIG. 5: 0.04 pM, 0.4 pM and 4 pM, or 4 pM, 40 pM and 400 pM Cy5-labeled anti-goat IgG, FIG. 6: 400 pM Cy3-labeled anti-rabbit antibody and 400 pM Cy5-labeled anti-goat antibody).

Chip Readout:

The fluorescence signal of the bound analyte molecules is detected in a commercial chip reader system from ArrayWorx. The exposure times range between 0.1 s and 2 s and are maintained constant within each experiment. The signal intensities are stored in the form of graduated gray values. The data is analyzed with the help of the Aida program from Raytest, Berlin, Germany.

### EXAMPLE 2

### Production of Nanoparticle Layers for MALDI TOF Analysis

Substrate:

Gold surfaces, in this case the MALDI target itself, are rubbed with acetone, treated with ultrasound for 3 minutes in 1:1 isopropanol/HCl (0.2 M), rinsed with isopropanol and blown dry with nitrogen. After this, they are incubated for 20 minutes at room temperature in an aqueous polyanion solution (0.02 M (in relation to the monomer) poly(acrylic acid) in MilliQ-H₂O), pH 8.5), washed for 5 minutes in MilliQ-H₂O, incubated for 20 minutes in a polycation solution (see Example 1), washed for another 5 minutes and blown dry with nitrogen.

Particles:

The silica particles are synthesized as described in Example 1 and then amino- and carboxy-functionalized.

Streptavidin Binding to Silica Particles:

Streptavidin in the amount of 2.68 nmol is added to 10 ml 0.1 m MES buffer (pH 5). To this, 5 mg of the carboxy particles are added. Then, 2 μmol of EDC is added. After shaking the mixture for 3 hours at room temperature, the particles are washed once with 10 ml MES buffer (pH 5) and once with 10 ml phosphate buffer (pH 7).

Preservation of the protein function in nanoparticle layers: In order to stabilize the function of nanoparticle-bound capture proteins in nanoparticle layers, the particles are suspended for coating in a 5% (w/v) aqueous trehalose solution.

Production of the Nanoparticle Layer:

Approximately 250 μg of streptavidin particles are suspended in 10 μl MilliQ-H₂O+5% trehalose and dried in pl batches on the substrate surface (approximately 2 mm²).

Use of Nanoparticle Surface:

First, the nanoparticle surfaces are blocked for one hour with a 3% (w/v) solution of BSA in PBS buffer, then incubated for 1.5 hours at room temperature with the analyte solution and thereafter washed for 30 minutes each in PBS/0.1% TritonX 100, PBS and MilliQ water.

The analyte solution is a mixture of single to triple biotinylated and unbiotinylated insulin dissolved in PBS buffer (approximately 3:1, total concentration approximately 250 nM).

Mass Spectrometry Analysis:

Matrix:

A saturated solution of 3,5-dimethoxy-4-hydroxy cinnamic acid was dissolved in 6:4 (v/v) 0.1% trifluoroacetic acid (p.A.) and acetonitrile (HPLC grade), placed on the particle layer and air-dried.

Hardware:

A mass spectrometer (HP G 2025A LD-TOF), which was modified with a time lag focusing (TLF) unit (Future, GSG company), was used. The data collection was carried out with a Le Croy 500 MHz oscilloscope.

1. A functional element, comprising a carrier having a surface and at least one microstructure on the carrier surface, the microstructure being formed by a plurality of layers of nanoparticles disposed three-dimensionally on top of each other and by the inclusion of at least one biomolecule-stabilizing agent, and the nanoparticles having molecule-specific recognition sites, which allow addressability within the microstructure.

2. The functional element according to claim 1, wherein the microstructure comprises at least one protein-stabilizing agent.

3. A functional element according to claim 1, wherein the three-dimensionally disposed layers have a thickness of 10 nm to 10 μm.

4. A functional element according to claim 3, wherein the protein-stabilizing agent is selected from the group consisting of a saccharide, polyalcohol, amino acid, polymer, organic or inorganic salt trimethylamine-N-oxide, sarcosine, betaine, gamma-aminobutyric acid, octopine, alanopine, strombine, dimethylsulfoxide, of ethanol or a mixture thereof.

5. A functional element according to claim 1, wherein the microstructure covers a surface area section of the carrier surface and at least one of the surface-to-length parameters of the covered surface section of the carrier surface is smaller than 999 μm and at least 10 nm.

6. A functional element according to claim 1, wherein the surface of the carrier comprises metal, metal oxide, polymer, semiconductor material, glass or ceramic material.

7. A functional element according to claim 1, wherein the surface of the carrier is planar or pre-structured.

8. A functional element according to claim 1, wherein the surface of the carrier comprises a layer of a chemical compound which prevents nonspecific deposition of biological molecules on the carrier surface.

9. A functional element according to claim 1, wherein a bonding agent layer is provided between the carrier surface and the microstructure.

10. A functional element according to claim 9, wherein the bonding agent is a polymer having chemically reactive groups.

11. The functional element according to claim 10, wherein the polymer is a hydrogel.

12. The functional element according to claim 9, wherein the bonding agent is a plasma layer comprising charged or uncharged chemically reactive groups or a self-assembled monolayer based on silane, mercaptan, phosphate or fatty acid.

13. A functional element according to claim 9, wherein the bonding agent is responsive to the pH value, the ion concentration or the temperature.

14. A functional element according to claim 1, wherein the nanoparticles comprise a core and a surface that has the molecule-specific recognition sites.

15. A functional element according to claim 14, wherein one or more biologically active molecules are bound to the molecule-specific recognition sites.

16. (canceled)

17. A functional element according to claim 15, wherein the bound molecules are selected from the group consisting of proteins, protein complexes, nucleic acids, PNA molecules, fragments thereof or a combination thereof.

18. A functional element according to claim 17, wherein the proteins are selected from the group consisting of antibodies, antigens, enzymes, cytokines, receptors and structural proteins.

19. A functional element according to claim 14, wherein the molecule-specific recognition sites comprise at least one first functional group and the bound molecules comprise complementary second functional groups that bind the first functional groups.

20. The functional element according to claim 19, wherein the first functional groups and the complementary second functional groups that bind the first functional groups are selected from the group consisting of active ester, alkylketone group, aldehyde group, amino group, carboxy group, epoxy group, maleinimido group, hydrazine group, hydrazide group, mercaptan group, thioester group, oligohistidine group, Strep-Tag I, Strep-Tag II, desthiobiotin, biotin, chitin, chitin derivative, chitin-binding domain, metal chelate complex, streptavidin, streptactin, avidin and neutravidin.

21. A functional element according to claim 19, wherein the first and second functional groups are molecular imprinted functional groups.

22. A functional element according to claim 19, wherein the at least one of the first and complementary second functional groups part of a spacer or connected by a spacer the surface of the nanoparticles.

23. (canceled)

24. A functional element according to claim 14, wherein the core of the nanoparticles comprises organic material.

25. The functional element according to claim 24, wherein the organic material is an organic polymer.

26. A functional element according to claim 25, wherein the organic polymer is polypropylene, polystyrene, polyacrylate or a mixture thereof.

27. A functional element according to claim 14, wherein the core comprises inorganic material.

28. A functional element according to claim 27, wherein the inorganic material is selected from the group consisting of a metal, SiO₂, SiO, a silicate, Al₂O₃, SiO₂·Al₂O₃, Fe₂O₃, Ag₂O, TiO₂, ZrO₂, Zr₂O₃, Ta₂O₅, zeolithe, glass, indium tin oxide, hydroxyl apatite, a Q-Dot or a mixture thereof.

29. A functional element according to claim 24, wherein the core measures between 5 nm and 500 nm in size.

30. A functional element according to claim 24, wherein the core comprises at least one function group.

31. A functional element according to claim 30, wherein the function is anchored in the core and is at least one member selected from the group consisting of a fluorescence marker, UV/Vis marker, a superparamagnetic function, a ferromagnetic function and a radioactive marker.

32. A functional element according to claim 30, wherein the surface of the core is modified with an organic or inorganic layer comprising the first functional groups, and the layer comprises a fluorescence marker, UV/Vis marker, a superparamagnetic function, a ferromagnetic function or a radioactive marker.

33. A functional element according to claim 30, wherein the surface of the core comprises a chemical compound, disposed so as to provide steric stabilization, to prevent conformation changes in the immobilized molecules or to prevent deposition of a further biologically active compound on the core.

34. The functional element according to claim 33, wherein the chemical compound is polyethylene glycol, oligoethylene glycol, dextrane or a mixture thereof.

35. A functional element according to claim 15, wherein the bound molecules comprise a marker.

36. A functional element according to claim 15, wherein further molecules are bound to the bound molecules.

37. A functional element according to claim 1, wherein a plurality of microstructures are disposed on the carrier surface, and the microstructures of comprise nanoparticles with different molecule-specific recognition sites.

38. The functional element according to claim 37, wherein different molecules are bound to the microstructures.

39. A functional element according to claim 1, the element being obtained by applying one or more microstructures to the carrier surface using a needle ring printer, a lithographic method, an ink jet technique, or a micro-contact printing method.

40. A method for producing a functional element according to claim 2, wherein at least one layer of a bonding agent and thereafter at least one three-dimensional multi-layer microstructure comprising nanoparticles with molecule-specific recognition sites are applied to the surface of a carrier, and wherein at least one protein-stabilizing agent is introduced in the microstructure beforehand, at the same time or subsequently.

41. A method according to claim 40, wherein the surface of the carrier is cleaned or activated, or both, before applying the bonding agent layer.

42. The method according to claim 41, wherein the carrier surface is chemically activated.

43. A method according to claim 42, wherein the carrier surface is charged.

44. A method according to claim 42, wherein the carrier surface is activated by applying a primer.

45. A method according to claim 42, wherein a self-assembly layer is applied to the carrier surface.

46. The method according to claim 41, wherein the carrier surface is activated by means of plasma.

47. A method according to claim 40, wherein a bonding agent layer is applied to the carrier surface, the layer being defined in terms of the shape and surface area thereof, and wherein the carrier is then immersed in a nanoparticle suspension, so that, as a result of the adhesion of the nanoparticles to the applied bonding agent layer, a microstructure that is defined in terms of the shape and surface area thereof is produced.

48. The method according to claim 47, wherein the bonding agent layer, which is defined in terms of the shape and surface area thereof is applied by means of a needle ring printer, a lithographic method, an ink jet method or a micro-contact printing method.

49. A method according to claim 40, wherein the carrier is immersed in a suspension or solution of the bonding agent, thus producing a bonding agent layer that covers the entire carrier surface, and wherein thereafter the nanoparticles are applied so as to produce a microstructure that is defined in terms of the shape and surface area thereof is produced.

50. The method according to claim 49, wherein the microstructure that is defined in terms of the shape and surface area thereof is applied by means of a needle ring printer, a lithographic method, an ink jet method or a micro-contact printing method.

51. (canceled)

52. A method according to claim 40, wherein before, after or before and after the nanoparticles are applied, biologically active molecules are bound to the molecule-specific recognition sites of the nanoparticles.

53. The method according to claim 52, wherein the biologically active molecules are bound to the molecule-specific recognition sites of the nanoparticles so that the molecule-specific recognition sites of the nanoparticles, which comprise first functional groups, are brought in contact with the molecules comprising complementary second functional groups that bind the first functional groups so that bonds are obtained between the functional groups of the molecule-specific recognition sites and the molecules.

54. A method according to claim 53, wherein the first functional groups and the complementary second functional groups that bind the first functional groups are selected from the group consisting of active ester, alkylketone group, aldehyde group, amino group, carboxy group, epoxy group, maleinimido group, hydrazine group, hydrazide group, mercaptan group, thioester group, oligohistidine group, Strep-Tag I, Strep-Tag II, desthiobiotin, biotin, chitin, chitin derivate, chitin-binding domain, metal chelate complex, streptavidin, streptactin, avidin and neutravidin.

55. (canceled)

56. A method according to claim 52, wherein the biologically active molecules are selected from the group consisting of proteins, protein complexes, antigens, nucleic acids, PNA molecules or fragments thereof.

57. In a method of analyzing an analyte by combining a sample with a detectable material and thereafter detecting the detectable material, the improvement which comprises utilizing a functional element according to claim 1 as the detectable material.

58. A method according to claim 57, wherein the detection method comprises MALDI mass spectrometry, fluorescence or UV/Vis spectroscopy, fluorescence or light microscopy, waveguide spectroscopy, impedance spectroscopy or another mass spectrometric, optical, gravimetric or electric method or a combination thereof.

59. In a method of controlling cell adhesion or cell growth in which the cell is combined with a control agent, the improvement which comprises utilizing as the control agent a functional element according to any claim 1.

60-61. (canceled)

62. In a method of diagnosing disease in which a material is combined with an entity in which the disease may be manifested, the improvement which comprises utilizing a functional element according to claim 1 as said material.

63. A method according to claim 62, wherein the is used for diagnosis comprises identifying pathogens.

64. A method according to claim 62, wherein diagnosis comprises identifying mutated genes in humans or animals.

65. A method according to claim 57, wherein the analyte is a microbiological contaminate in the sample.

66. A method according to claim 65, wherein the sample is a water or soil sample.

67. A method according to claim 65, wherein the sample is a food or animal feed sample.

68. A biocomputer containing an electronic component which comprises a functional element according to claim 1.

69. A method for identifying or detecting an analyte in a solution or suspension, or both, wherein in a first step a) a functional element according to claim 1 is provided, subsequently in a second step b) the functional element is brought in contact with the solution or suspension comprising the analyte, thereafter in a third step c) non-bound analyte is removed from the functional element by means of a biocompatible washing fluid and subsequently in a fourth step d) a detection method is carried out.

70. (canceled)

71. A method according to claim 69, wherein the detection method carried out in step d) is a fluorescence detection method or a MALDI mass spectrometry method.

72. The method according to claim 71, wherein the detection method that is carried out is a fluorescence detection method and wherein the analyte or the bound biological molecule, or both, are fluorescence labeled.
